(19) United States
(12) Patent Application Publication
(10) Pub. No.: US 2011/0297820 A1
(43) Pub. Date: Dec. 8, 2011

(54) PSEUDACYCLIN AND METHOD OF INDICATION OF A FUNGUS PSEUDALLESCHERIA BOYDII

(76) Inventors: Vladimir Havlicek, Praha (CZ); Marek Kuzma, Plzen (CZ); Jan Sklenar, Praha (CZ); Martin Zabka, Praha (CZ); Jan Nedved, Plana Nad Luznici (CZ); Petr Novak, Dolni Brezany (CZ); Karel Lemr, Olomouc (CZ)

(21) Appl. No.: 12/992,346

(22) PCT Filed: Jun. 10, 2009
(86) PCT No.: PCT/CZ2009/000081

§ 371 Date: Dec. 9, 2010

(30) Foreign Application Priority Data
Jun. 10, 2008 (CZ) PV 22008 353

(51) Int. Cl. H01J 49/26; C07K 7/56

(52) U.S. Cl. 250/282; 530/317

(57) ABSTRACT

Pseudacyclin and method of indication of a fungus Pseudallescheria boydii Pseudacyclins are secondary metabolites isolated from a filamentous fungus Pseudallescheria boydii. They reveal a cyclic-like structure which is represented by a cyclopeptide backbone consisting of an amino acid sequence: ornithine-phenylalanine-proline-isoleucine-the fifth amino acid. This cycle is completed by a peptide bond condensed from an amino group on a C^{δ} of ornithine and a carboxyl group of the fifth amino acid, which is variable. The peptides further contain a side chain represented by a second variable residue, which is the sixth amino acid bound to the cyclopeptide skeleton. The attachment is made through a peptide bond originated from the condensation of the amino group at C^{α} of ornithine and carboxyl group of this sixth amino acid residue. Pseudacyclins as specific products of secondary metabolism of a pathogenic filamentous fungus Pseudallescheria boydii are useful as disease markers. Pseudacyclins are used as standards indicating the fungal presence in an organism.

### TECHNICAL FIELD

The structures of specific secondary metabolites of a fungus Pseudallescheria boydii which are important for the identification of an infectious disease.

### BACKGROUND ART

Pseudallescheria boydii is a microscopic filamentous fungus causing infectious fungal diseases. It is an opportunistic microbial pathogen of a biohazard safety level-2 (BSL-2) category.

For an effective medical treatment it is important to identify the microbial species which has caused the infection. Based on the symptoms, the infections caused by Pseudallescheria are often misplaced by those caused by different Aspergilli strains that are sensitive to different antifungal drugs. Recently, the clinical mycology has just a few and restricted diagnostic tools for this particular disease. These are nonspecific RTG and CT scans, some experimental laboratories carry out the analyses of fungal RNA in patient's blood or tissue. Further, insensitive and nonspecific diagnostic sets based on sacccharidic composition of a fungal cell are sometimes used.

There is no specific diagnostic tool for the determination of an infectious disease caused by pathogenic microorganism Pseudallescheria boydii. Hence, a targeted treatment cannot be used.

### DISCLOSURE OF THE INVENTION

The precise and specific identification of an infectious disease caused by a pathogenic microorganism Pseudallescheria boydii is represented by the detection of its specific secondary metabolites. Secondary metabolism uses a sophisticated multienzyme system which is mostly activated once the fungal body growth is terminated. If a specific secondary metabolite is found in the host body, the existence of a particular microorganism is thus confirmed and targeted treatment can be used. In the case of Pseudallescheria boydii five specific secondary metabolites have been identified in the course of a fungal life cycle indicating thus the pathogen presence in the host.

Cyclic peptides bearing a side chain were isolated from a fermentation broth obtained by a cultivation of a filamentous fungus Pseudallescheria boydii. Chemical structures have been determined with these peptides. Five structures exert four conserved amino acid positions and two positions with variable amino acid composition. The general structural feature is represented by a cyclopeptide backbone consisting of an amino acid sequence: ornithine-phenylalanine-proline-isoleucine-the fifth amino acid. This cycle is completed by a peptide bond condensed from an amino group on a C^{δ} of ornithin and carboxy group of the fifth amino acid. The side chain consists either of an N-acetyl-isoleucyl, N-acetyl-methyl-isoleucyl, or N-acetyl-valyl, that are bound to the cyclopeptide skeleton through a peptide bond originated from the condensation of the amino group at C^{α} of ornithine and carboxyl group of the sixth amino acid. The structures also differ in the composition of the fifth amino acid. Pseudacyclin A contains isoleucine as the fifth amino acid. Pseudacyclin B contains valine at this 5th position. On the contrary, Pseudacyclin C contains N-methyl-acetyl-isoleucyl at the 6^{th} position. Pseudacyclin D contains valine at the 6^{th} position. Pseudacyclin E contains valines both at the 5^{th} and 6^{th} positions, i.e. has two amino acid residues replaced when compared to Pseudacyclin A.

For the preparation of secondary metabolites the micromycete Pseudallescheria boydii was cultivated for 21 days. Low-molecular weight peptides were isolated from the fermentation broth by separation techniques. The peptides were extracted from media by dichloromethane; the extracts were dried on a speedvac concentrator and finally, the sample was re-dissolved in methanol. Peptides were also purified by solid-phase extraction; enriched peptides were eluted by methanol from the corresponding cartridge. Concentrated peptides were then subjected to high performance liquid chromatography: the separation was carried out on a reversed-phase column and the eluted peptides were detected spectrophotometrically at 214 nm. The content and nature of separated fractions was determined by mass spectrometry.

The structure of Pseudacyclin A was determined by nuclear magnetic resonance at the following conditions: ¹Ha ¹³C NMR (399.87 MHz a 100.55 MHz, CD3CN, 30° C.), *gHSQC, +2 C.

### FIGURES

FIG. 1: The structure of Pseudacyclin A.

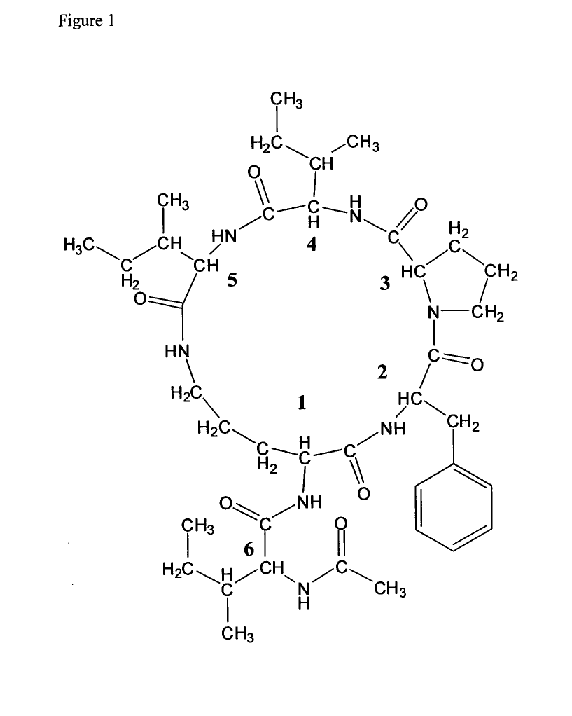

FIG. 2: The structure of Pseudacyclin B.

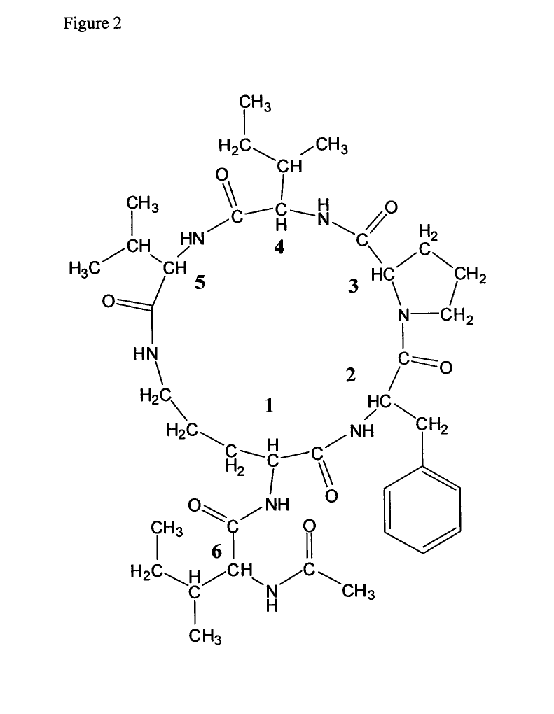

FIG. 3: The structure of Pseudacyclin C.

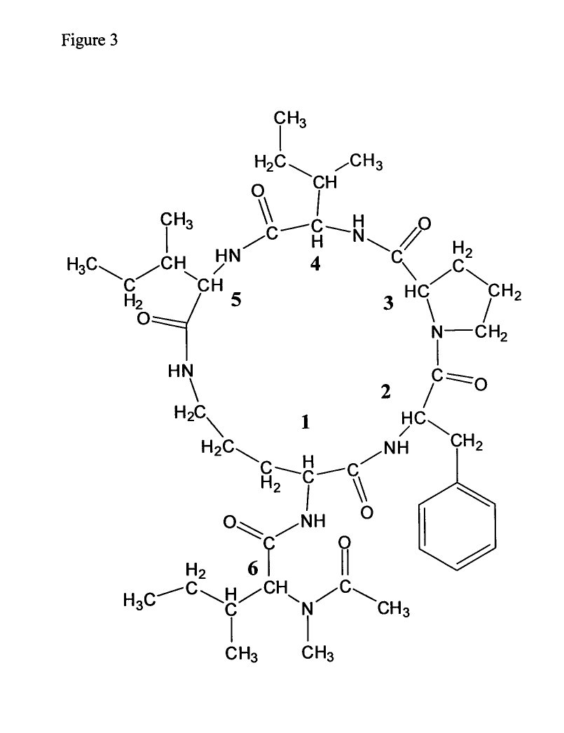

FIG. 4: The structure of Pseudacyclin D.

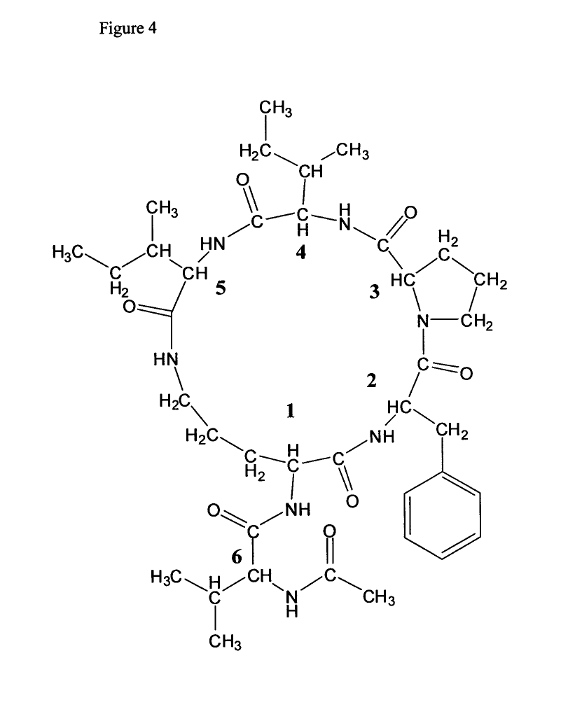

FIG. 5: The structure of Pseudacyclin E.

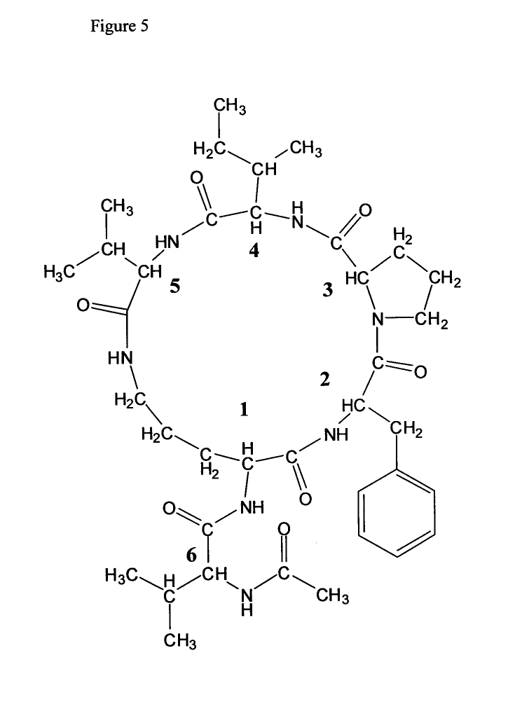

FIG. 6: Table of identified structures.

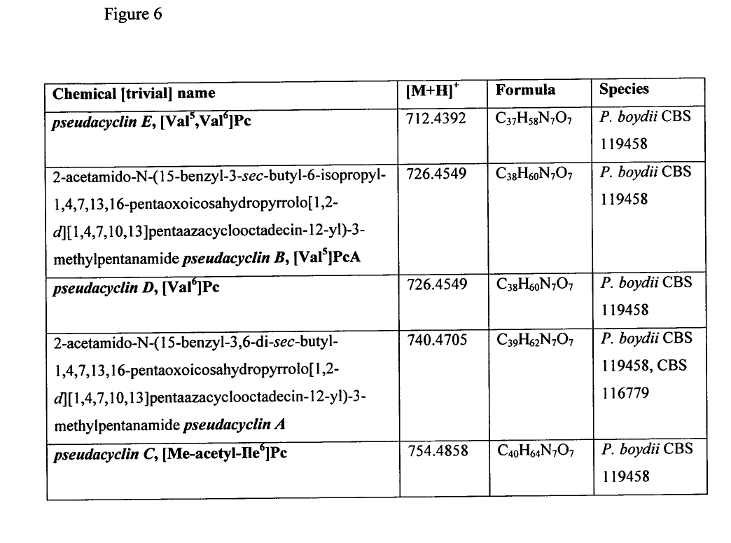

FIG. 7: Table indicating the determination of Pseudacyclin A by nuclear magnetic resonance spectroscopy.

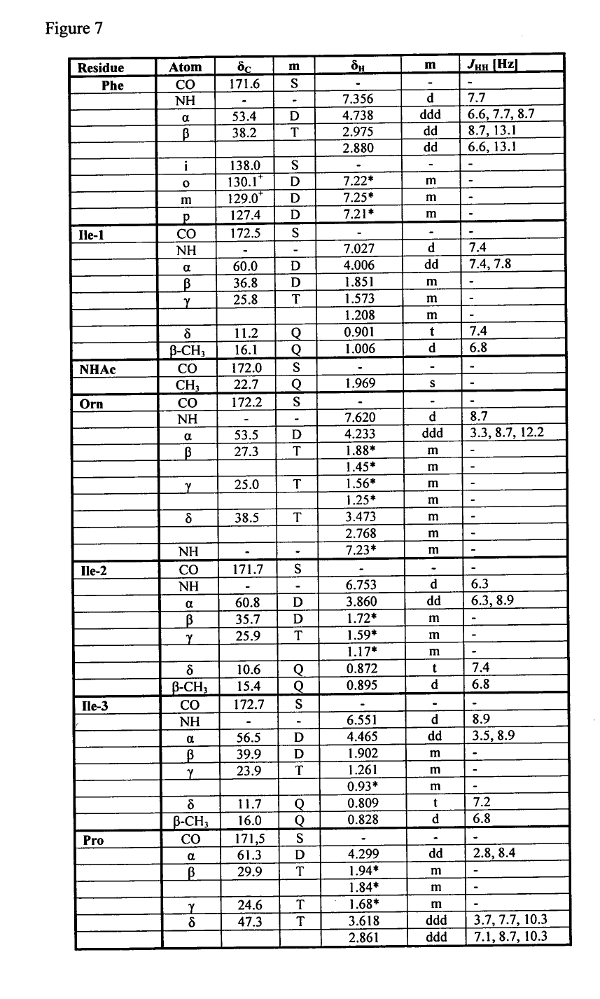

### EXAMPLES

### Example 1

Pseudacyclin A is a low-molecular peptide containing a cyclopeptide skeleton of an amino acid sequence ornithine-phenylalanine-proline-isoleucine-fifth amino acid. The cycle is completed by a peptide bond condensed from an amino group on a C^{δ} of ornithine and carboxyl group of the fifth amino acid, which is isoleucine in this case. The peptide further contains a side chain represented by N-acetyl-isoleucyl, which is the sixth amino acid bound to the cyclopeptide skeleton. The attachment is made through a peptide bond originated from the condensation of the amino group at C^{α} of ornithine and carboxyl group of this N-acetyl-isoleucine.

### Example 2

Pseudacyclin B is a low-molecular peptide containing a cyclopeptide skeleton of an amino acid sequence ornithine-phenylalanine-proline-isoleucine-fifth amino acid. The cycle is completed by a peptide bond condensed from an amino group on a C^{δ} of ornithine and carboxyl group of the fifth amino acid, which is valine in this case. The peptide further contains a side chain represented by N-acetyl-isoleucyl, which is the sixth amino acid bound to the cyclopeptide skeleton. The attachment is made through a peptide bond originated from the condensation of the amino group at C^{α} of ornithine and carboxyl group of this N-acetyl-isoleucine.

### Example 3

Pseudacyclin C is a low-molecular peptide containing a cyclopeptide skeleton of an amino acid sequence ornithine-phenylalanine-proline-isoleucine-fifth amino acid. The cycle is completed by a peptide bond condensed from an amino group on a C^{δ} of ornithine and carboxyl group of the fifth amino acid, which is isoleucine in this case. The peptide further contains a side chain represented by N-acetyl-N-methyl isoleucyl, which is the sixth amino acid bound to the cyclopeptide skeleton. The attachment is made through a peptide bond originated from the condensation of the amino group at C^{α} of ornithine and carboxyl group of this N-acetyl-N-methyl-isoleucine.

### Example 4

Pseudacyclin D is a low-molecular peptide containing a cyclopeptide skeleton of an amino acid sequence ornithine-phenylalanine-proline-isoleucine-fifth amino acid. The cycle is completed by a peptide bond condensed from an amino group on a C^{δ} of ornithine and carboxyl group of the fifth amino acid, which is isoleucine in this case. The peptide further contains a side chain represented by N-acetyl-valyl, which is the sixth amino acid bound to the cyclopeptide skeleton. The attachment is made through a peptide bond originated from the condensation of the amino group at C^{α} of ornithine and carboxyl group of this N-acetyl-valine.

### Example 5

Pseudacyclin E is a low-molecular peptide containing a cyclopeptide skeleton of an amino acid sequence ornithine-phenylalanine-proline-isoleucine-fifth amino acid. The cycle is completed by a peptide bond condensed from an amino group on a C^{δ} of ornithine and carboxyl group of the fifth amino acid, which is valine in this case. The peptide further contains a side chain represented by N-acetyl-N-methylvalyl, which is the sixth amino acid bound to the cyclopeptide skeleton. The attachment is made through a peptide bond originated from the condensation of the amino group at C^{α} of ornithine and carboxyl group of this N-acetyl-N-methyl-valine.

### Example 6

Pseudacyclins have been received by a submerged cultivation of a fungus Pseudallescheria boydii at 28° C. and 280 cycles/minute. The cultivation started by an inoculating the 150 mL medium with 3×10⁷ fungal spores. The composition of the inoculum was saccharose 30 g, maltose extract 20 g, KH₂PO₄ 2 g, H₂O 1000 ml. After 72 hours the inoculum cultivation was stopped and 25 mL of inoculum was used for inoculating of 250 mL of production media. The medium with reduced saccharide content was determined as the optimum cultivation medium. Its composition was NaNO₃ 2 g, K₂HPO₄ 1 g, KCl 0.5 g, MgSO₄.7H2O 0.5 g, FeSO₄ 0.01 g, saccharose 10 g, H₂O 1000 ml, pH adjusted to 7.3. The peptide production phase of the cultivation was terminated in 21 days. The mycelium was separated from the fermentation broth and the culture medium filtrate has been prepared this way.

Isolation and Purification of the Produced Peptides

Liquid-Liquid Extraction of the Peptides

The culture filtrate (50 mL) was extracted twice by dichloromethane (50 mL). Organic layers were pooled, separated and dried. The dry residue was re-constituted in methanol (0.2 mL) and diluted in HPLC solvent A (10× dilution, for composition see below).

Solid-Phase Extraction

The resulting solution was injected on a Strata C18E column (1 g, Phenomenex) and desalted by 5% aqueous methanol. The cartridge was then stepwise eluted with 20% (step 1), 50% (step 2), and 100% (step 3) aqueous methanol. In each step ten fractions of 7 ml each were collected and evaporated in a speedvac. The fractions were subsequently examined for their metabolite contents. The step 1 (fraction 1) removed the UV-absorbing yellow-to-brownish interfering substances (possibly melanine structures). Steps 2 and 3 yielded cyclic peptides with lasso structures.

Preparative Chromatography

The produced peptides were purified on a reversed phase Watrex WRP-18 (7 μm) preparative column with a gradient elution (A: 5% acetonitrile/water with 0.05% trifluoroacetic acid and B: 95% acetonitrile/water with 0.05% trifluoroacetic acid, all Merck, gradient grade) at 8 mL/min flow rate and 40° C. Gradient profile started with 50 minute hold at 0% B and continued with 0-12% B in 5 min; 12-70% B in 150 min, 70-100% B in 25 min and 20 min hold at 100% B. Separated peptides were detected by UV (214 nm) and subjected to spectral analyses.

LC-MS Experimental Conditions

Fungal extract was loaded on a chromatographic column (Magic Michrom BioResources, C18, 5 um, 0.180 mm×100,000 mm; flow rate 0.001 ml/min). The peptides were separated with a gradient elution (A: 5% acetonitrile/water with 0.5% acetic acid and B: 95% acetonitrile/water with 0.5% trifluoroacetic acid, all Merck, gradient grade). Gradient profile started with 5 minute hold at 0% B and continued with 0-100% B in 30 min; and 15 min hold at 100% B. Peptides were eluted at 40-70% B and their sequences were deduced from MS/MS mass spectra.

Pseudacyclin A HR-MS/MS: 740.4705, [M+H]⁺, C₃₉H₆₂N₇O₇; 712.4761, [MH—CO]⁺, C₃₈H₆₂N₇O₆; 585.3760, core, C₃₁H₄₉N₆O₅; 471.2967, ¹⁻²B₄, C₂₆H₃₉N₄O₄; 443.3018, C₂₅H₃₉N₄O₃, ¹⁻²A₄; 358.2125, ¹⁻²B₃, C₂₀H₂₈N₃O₃; 324.2280, ²⁻³B₃, C₁₇H₃₀N₃O₃; 270.1810, C₁₃H₂₄N₃O₃.

Pseudacyclin B, HR-MS/MS: 726.4549, [M+H]⁺; 698.4594, [MH—CO]⁺; 571.3601, core; 457.2808, ¹⁻²B₄; 429.2858, ¹⁻²A₄; 358.2123, ¹⁻²B₃; 310.2125, ²⁻³B₃; 270.1812.

Pseudacyclin C, HR-MS/MS: 754.4858, [M+H]⁺, 726.4903, [MH—CO]⁺; 599.3907, core; 471.2961, ¹⁻²B₄; 443.3012, ¹⁻²A₄; 358.2122, ¹⁻²B₃; 324.2279, ²⁻³B₃; 284.1966

Pseudacyclin D, MS/MS: 726.4549, [M+H]⁺; 698.2, [MH—CO]⁺; 585.2, core; 471.1, ¹⁻²B₄; 443.1, ¹⁻²A₄; 358.0, ¹⁻²B₃; 324.2, ²⁻³B₃.

Pseudacyclin E, MS/MS: 712.4401, [M+H]⁺; 684.4, [MH—CO]⁺; 571.2, core; 457.1, ¹⁻²B₄; 429.1, ¹⁻²A₄; 358.2, ¹⁻²B₃; 310.0, ²⁻³B₃.

### Example 7

The mixture of isolated peptides from bodily fluids is deposited on MALDI or DESI target of a mass spectrometer and the corresponding spectrum is recorded. The acquired mass spectrum is compared with that of standard pseudacyclin.

### INDUSTRIAL APPLICABILITY

The isolated pseudacyclins are useful in pharmaceutical industry as antigens for the preparation of specific antibodies and for the production of diagnostic immunoanalytical sets based on the cross-reactivity antigen-antibody for the diagnosing of an infectious disease caused by Pseudallescheria boydii.

Pseudacyclins can be used in medicine as standards for diagnosis of this disease by means of mass spectrometry and specific immunoanalytical methods.

1. Pseudacyclin, isolated from a fungus Pseudallescheria boydii, consisting of a cyclic structure with a side chain, made by five amino acid residues forming a sequence Orn-Phe-Pro-Ile-fifth amino acid residue, which is completed by a peptide bond condensed from an amino group on a C^{δ} of ornithine and a carboxyl group of the fifth amino acid residue and a side chain represented by a sixth amino acid residue, which is bound to the cyclopeptide skeleton through a peptide bond originated from the condensation of the amino group at C^{α} of ornithine and carboxyl group of the sixth amino acid residue.

2. Pseudacyclin according to claim 1, whereof the fifth amino acid residue is isoleucine and sixth amino acid residue is N-acetyl-isoleucine.

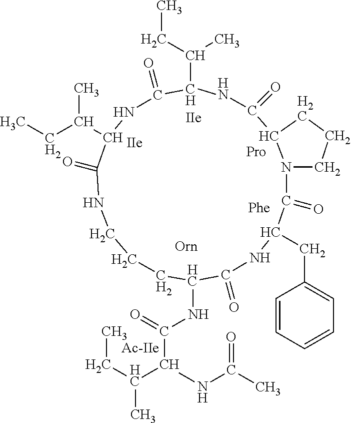

3. Pseudacyclin according to claim 1, whereof the fifth amino acid residue is valine and sixth amino acid residue is N-acetyl-isoleucine.

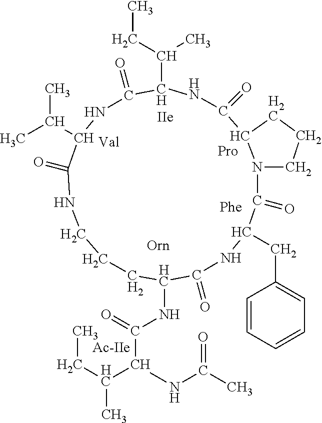

4. Pseudacyclin according to claim 1, whereof the fifth amino acid residue is isoleucine and sixth amino acid residue is N-acetyl-N-methyl-isoleucine.

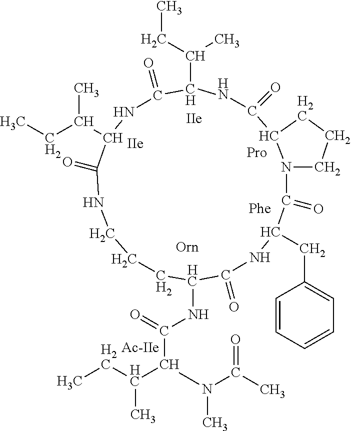

5. Pseudacyclin according to claim 1, whereof the fifth amino acid residue is isoleucine and sixth amino acid residue is N-acetyl-valine.

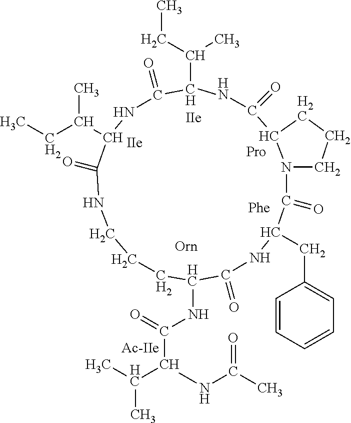

6. Pseudacyclin according to claim 1, whereof the fifth amino acid residue is valine and sixth amino acid residue is N-acetyl-valine.

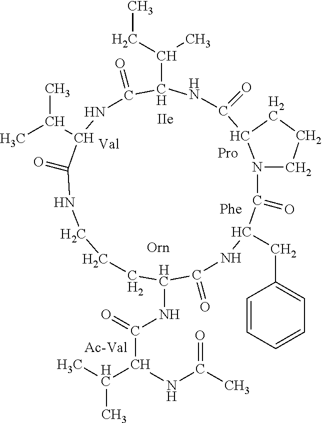

7. The method of indication of a fungus Pseudallescheria boydii in an organism characterized in that low-molecular peptides isolated from bodily fluids are analyzed by mass spectrometry and their spectrum are compared with mass spectrum of Pseudacyclin according to claims 2-6 and are searched for identical structures.
